(19) United States
(12) Patent Application Publication
(10) Pub. No.: US 2011/0022981 A1
(43) Pub. Date: Jan. 27, 2011

(54) PRESENTATION OF DEVICE UTILIZATION AND OUTCOME FROM A PATIENT MANAGEMENT SYSTEM

(76) Inventors: Deepa Mahajan, Circle Pines, MN (US); Yanting Dong, Shoreview, MN (US); Christopher Monnier, Eden Prairie, MN (US); Nicholas Everhart, Minneapolis, MN (US); Paul Jones, St. Paul, MN (US); Milan Seth, Minneapolis, MN (US); Haresh G. Sachanandani, Culver City, CA (US); Kenneth P. Hoyme, Plymouth, MN (US)

Correspondence Address: SCHWEGMAN, LUNDBERG & WOESSNER/BSC-CRM, PO BOX 2938, MINNEAPOLIS, MN, 55402, US

(21) Appl. No.: 12/840,611

(22) Filed: Jul. 21, 2010

Related U.S. Application Data
(60) Provisional application No. 61/228,112, filed on Jul. 23, 2009.

(51) Int. Cl. G06F 3/048; A61B 5/00

(52) U.S. Cl. 715/810; 600/300

(57) ABSTRACT

In a graphical user interface displayed in an electronic display of a computing device, a first device profile and second device profile are presented, the first and second device profiles each comprising at least one device parameter used to configure a medical device of a subject. A user input control is presented to select one of the first or second device profiles to provide a selected device profile. A probabilistic outcome of the subject corresponding to the selected device profile is then presented.

### CROSS-REFERENCE TO RELATED APPLICATION

This application claims the benefit of U.S. Provisional Application No. 61/228,112, filed on Jul. 23, 2009, under 35 U.S.C. §119(e), the benefit of priority of which is claimed herein, and which is incorporated herein by reference in its entirety.

### TECHNICAL FIELD

This document pertains generally to implantable medical devices, and more particularly, but not by way of limitation, to presentation of device utilization and outcomes.

### BACKGROUND

Medical devices are used to manage patient conditions. Some medical devices are used for cardiac rhythm or function management. Cardiac rhythm or function management devices can include implantable devices that monitor or maintain heart rhythm or function. These types of devices can include pacers, defibrillators, cardioverters, cardiac resynchronization therapy (CRT), or various combinations of these or other devices. A cardiac rhythm or function management devices can be used to sense intrinsic heart contractions, deliver pacing pulses to evoke responsive heart contractions, or deliver a shock to interrupt certain arrhythmias.

Modern medical devices include programmable elements. For example, in the context of a pacing device, various parameters such as pacing amplitude, pacing rate, and pulse width can be configured or adjusted by a clinician or other care provider. In some cases, a large number of configurable parameter are available and it is not always clear how changes to one parameter may affect other parameter; or how the changes to one parameter may affect a projected outcome of a patient.

### OVERVIEW

In a graphical user interface displayed in an electronic display of a computing device, a first device profile and second device profile are presented, the first and second device profiles each comprising at least one device parameter used to configure a medical device of a subject. A user input control is presented to select one of the first or second device profiles to provide a selected device profile. A probabilistic outcome of the subject corresponding to the selected device profile is then presented.

Example 1 describes a system comprising an electronic display; a memory to store a first and second device profile, each profile comprising at least one device parameter used to configure a medical device of a subject; and a processor, coupled to the memory and the electronic display, the processor configured to: present, in a graphical user interface displayed in the electronic display, the first device profile and second device profile; present, in the graphical user interface, a user input control to select one of the first or second device profiles to provide a selected device profile; and present, in the graphical user interface, a probabilistic outcome of the subject corresponding to the selected device profile.

In Example 2, the system of Example 1 is optionally configured to present, in the graphical user interface, a user input control to configure the medical device using the selected device profile; and in response to the user input control being activated, configure the medical device with the selected device profile.

In Example 3, the systems of Example 1 or 2 optionally include a patient monitor operatively coupled to the processor, where the patient monitor is configured to determine an efficacy of the medical device when configured with the selected device profile; compare the efficacy of the medical device to a target outcome; and when the efficacy of the medical device deviates more than a threshold value from the target outcome, communicating an alert.

In Example 4, the systems of any one or more of Examples 1-3 optionally configured such that the first device profile is a current device profile of the medical device and the second device profile is a recommended device profile for the medical device.

In Example 5, the system of any one or more of Examples 1-4 are optionally configured such that the processor is configured to obtain the recommended device profile from a database of device profiles.

In Example 6, the system of any one or more of Examples 1-5 are optionally configured to present a user input control to adjust a parameter associated with the selected device profile; and in response to the user input control being used, dynamically revise the probabilistic outcome to provide a revised outcome; and present the revised outcome in the graphical user interface.

In Example 7, the system of any one or more of Examples 1-6 are optionally configured to: present a user input control to select the probabilistic outcome from a plurality of available probabilistic outcomes, wherein the plurality of available probabilistic outcomes are selected based on at least one of the first or second device profiles.

In Example 8, the system of any one or more of Examples 1-7 are optionally configured to identify the medical device to provide a medical device identification; and use the medical device identification to selectively present the plurality of available probabilistic outcomes.

In Example 9, the system of any one or more of Examples 1-8 are optionally configured to: present a user input control to select a second probabilistic outcome; and present, in the graphical user interface, the second probabilistic outcome of the subject corresponding to the selected device profile.

In Example 10, the system of any one or more of Examples 1-9 are optionally configured such that the second probabilistic outcome is presented with the first probabilistic outcome.

Example 11 describes a method comprising: presenting, in a graphical user interface displayed in an electronic display of a computing device, a first device profile and second device profile, the first and second device profiles each comprising at least one device parameter used to configure a medical device of a subject; presenting, in the graphical user interface, a user input control to select one of the first or second device profiles to provide a selected device profile; and presenting, in the graphical user interface, a probabilistic outcome of the subject corresponding to the selected device profile.

In Example 12, the method of Example 11 is optionally performed comprising: presenting, in the graphical user interface, a user input control to configure the medical device using the selected device profile; and in response to the user input control being activated, configuring the medical device with the selected device profile.

In Example 13, the methods of Examples 11 or 12 are optionally performed such that the first device profile is a current device profile of the medical device and the second device profile is a recommended device profile for the medical device.

In Example 14, the methods of any one or more of Examples 11-13 are optionally performed comprising obtaining the recommended device profile from a database of device profiles.

In Example 15, the methods of any one or more of Examples 11-14 are optionally performed comprising: presenting a user input control to adjust a parameter associated with the selected device profile; and in response to the user input control being used, dynamically revising the probabilistic outcome to provide a revised outcome; and presenting the revised outcome in the graphical user interface.

In Example 16, the methods of any one or more of Examples 11-15 are optionally performed comprising: presenting a user input control to select the probabilistic outcome from a plurality of available probabilistic outcomes, wherein the plurality of available probabilistic outcomes are selected based on at least one of the first or second device profiles.

In Example 17, the methods of any one or more of Examples 11-16 are optionally performed comprising: identifying the medical device to provide a medical device identification; and using the medical device identification to selectively present the plurality of available probabilistic outcomes.

In Example 18, the methods of any one or more of Examples 11-17 are optionally performed comprising: presenting a user input control to select a second probabilistic outcome; and presenting, in the graphical user interface, the second probabilistic outcome of the subject corresponding to the selected device profile.

In Example 19, the methods of any one or more of Examples 11-18 are optionally performed such that the second probabilistic outcome is presented with the first probabilistic outcome.

Example 20 describes a machine-readable medium including instructions, which when executed on a machine, cause the machine to: present, in a graphical user interface displayed in an electronic display of the machine, a first device profile and second device profile, the first and second device profiles each comprising at least one device parameter used to configure a medical device of a subject; present, in the graphical user interface, a user input control to select one of the first or second device profiles to provide a selected device profile; and present, in the graphical user interface, a probabilistic outcome of the subject corresponding to the selected device profile.

Example 21 describes a system comprising: an electronic display; a user input device coupled to the electronic display; means for presenting, in a graphical user interface displayed in the electronic display of the machine, a first device profile and second device profile, the first and second device profiles each comprising at least one device parameter used to configure a medical device of a subject; means for presenting, in the graphical user interface, a user input control accessible with the user input device, the user input control to select one of the first or second device profiles to provide a selected device profile; and means for presenting, in the graphical user interface, a probabilistic outcome of the subject corresponding to the selected device profile.

This overview is intended to provide an overview of subject matter of the present patent application. It is not intended to provide an exclusive or exhaustive explanation. The Detailed Description is included to provide further information about the present patent application.

### BRIEF DESCRIPTION OF THE DRAWINGS

In the drawings, which are not necessarily drawn to scale, like numerals may describe similar components in different views. Like numerals having different letter suffixes may represent different instances of similar components. [0004]

Some embodiments are illustrated by way of example, and not limitation, in the figures of the accompanying drawings in which:

FIG. 1 is a schematic diagram illustrating a network system;

FIG. 2 is a flow chart illustrating a method for presenting information to assist a clinician when programming a medical device;

FIGS. 3-6 are diagrams illustrating example user interfaces;

FIGS. 7 and 8 are diagrams illustrating population-based views of parameter usage;

FIG. 9 is a diagram illustrating a temporal view of parameter usage; and

FIG. 10 is a block diagram illustrating a machine in the example form of a computer system, within which a set or sequence of instructions for causing the machine to perform any one of the methodologies discussed herein may be executed, according to various embodiments.

### DETAILED DESCRIPTION

Modern medical devices can include processing and storage components that provide programming capabilities. Programming can be used to provide therapy routines in an effort to increase a patient's life expectancy or reduce patient discomfort. At a low level, programming parameters can be used to control aspects of a medical device, such as pacing amplitude, pacing rate, and pulse width. At a high level, programming parameters can be used to control algorithmic decision making or algorithm selection. For example, algorithmic decision making can be used to increase arrhythmia discrimination accuracy, provide an appropriate treatment once an arrhythmia is detected, or communicate alerts under certain circumstances.

Efficient and effective programming is difficult for several reasons. Parameters that may be individually adjusted do not necessarily operate in isolation. In many cases, one parameter can have a dependent relationship with one or more other parameters, such that adjusting one parameter in isolation may cause a less desirable programming profile, and ultimately less effectively impact a patient's potential outcome. Moreover, the potential effects of adjusting one or more parameters are difficult to foresee. By using a statistical population and various correlation and association processes, a probabilistic outcome can be calculated and presented to the clinician at the time of programming to aid in the selection of programming parameters. For example, a user interface can be implemented to present one or more patient outcomes that are dynamically updated based on selected parameter values. A user interface like this can be used in a clinical setting, a research setting, or an educational setting. For example, clinicians can be provided a statistical basis to support programming changes; researchers can use a user interface like this with historical or simulated data to observe and study various programming profiles; and educators can use an interface like this to train clinicians and technicians. In addition, the interface can be used in a multitude of environments, ranging from a stand-alone application environment to a network-based application setting.

For the purposes of this document, a probabilistic outcome is a patient outcome that is determined using one or more statistical functions. A probabilistic outcome does not necessarily produce a probability of an outcome occurring, but is rather based on a probabilistic determination.

A device profile is a set of one or more parameters used to configure a device. A device profile may be stored, for example, in a database. A device profile may also be formed by any contemporaneous set of parameters. While a contemporaneous set of parameters may be downloaded to a device to program the device—this is not always the case. In fact, in some cases, a contemporaneous device profile is ephemeral in nature, such as when a user is comparing various available device profiles when considering a programming decision.

A user input control is a graphical element that, when activated, produces an programmatic event. The programmatic event can be detected, trapped, identified, or otherwise used to trigger subsequent processing. User input controls include, but are not limited to, buttons, icons, check boxes, radio buttons, hyperlinks, script controls, and the like. The user input control may be activated by various means, including but not limited to, a keyboard action, a pointing device action, a touch screen action, a voice command, or other user inputs.

The examples described herein include systems and methods for presenting a probabilistic outcome for a programming profile.

### System Overview

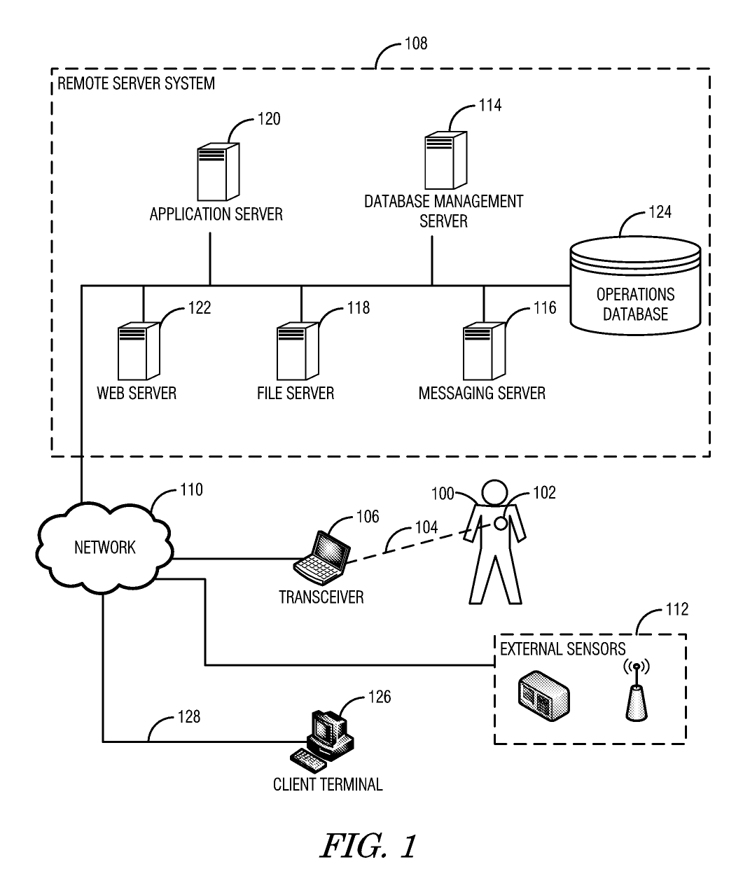

FIG. 1 illustrates portions of a system that enables physician-patient communication. In the example of FIG. 1, a patient 100 is provided with an implantable medical device (IMD) 102. Examples of implantable medical devices include a pacemaker, an implantable cardioverter defibrillator (ICD), a cardiac resynchronization therapy pacemaker (CRT-P), a cardiac resynchronization therapy defibrillator (CRT-D), a neurostimulation device, a deep brain stimulation device, a cochlear implant or a retinal implant. In some examples, the IMD 102 is capable of sensing physiological data and storing such data for later communication. Examples of physiological data include implantable electrograms, surface electrocardiograms, heart rate intervals (e.g., AA, VV, AV or VA intervals), electrogram templates such as for tachyarrhythmia discrimination, pressure (e.g., intracardiac or systemic pressure), oxygen saturation, activity, heart rate variability, heart sounds, impedance, respiration, intrinsic depolarization amplitude, or the like.

The IMD 102 is capable of bidirectional communication using a connection 104 with a computing device 106. A computing device is a device capable of receiving input, processing instructions, storing data, presenting data in a human-readable form, and communicating with other devices. The IMD 102 receives commands from the computing device 106 and may also communicate one or more patient indications to the computing device 106. Examples of patient indications include sensed or derived measurements such as heart rate, heart rate variability, data related to tachyarrhythmia episodes, hemodynamic stability, activity, therapy history, autonomic balance motor trends, electrogram templates for tachy discrimination, heart rate variability trends or templates, or trends, templates, or abstractions derived from sensed physiological data. Patient indications include one or more physiological indications, such as the physiological data described above. The IMD 102 may also communicate one or more device indications to the computing device 106. Examples of device indications include lead/shock impedance, pacing amplitudes, pacing thresholds, or other device metrics. In certain examples, the IMD 102 may communicate sensed physiological signal data to the computing device 106, which may then communicate the signal data to a remote device for processing.

Typically, the computing device 106 is located in close proximity to the patient 100. The computing device 106 may be attached, coupled, integrated or incorporated with a personal computer or a specialized device, such as a medical device programmer. In an example, the computing device 106 is a hand-held device. In examples, the computing device 106 is a specialized device or a personal computer. In an example, the computing device 106 is adapted to communicate with a remote server system 108. The communication link between the computing device 106 and the remote server system 108 is made through a computer or telecommunications network 110. The network 110 may include, in various examples, one or more wired or wireless networking such as the Internet, satellite telemetry, cellular telemetry, microwave telemetry, or other long-range communication networks.

In an example, one or more external sensors 112 are adapted to communicate with the computing device 106 or the remote server system 108 and may transmit and receive information, such as sensed data. External sensors 112 may be used to measure patient physiological data, such as temperature (e.g., a thermometer), blood pressure (e.g., a sphygmomanometer), blood characteristics (e.g., glucose level), body weight, physical strength, mental acuity, diet, or heart characteristics. An external sensor 112 may also include one or more environmental sensors. The external sensors 112 can be placed in a variety of geographic locations (in close proximity to patient or distributed throughout a population) and can record non-patient specific characteristics such as, for example, temperature, air quality, humidity, carbon monoxide level, oxygen level, barometric pressure, light intensity, and sound.

External sensors 112 can also include devices that measure subjective data from the patient. Subjective data includes information related to a patient's feelings, perceptions, and/or opinions, as opposed to objective physiological data. For example, the “subjective” devices can measure patient responses to inquiries such as “How do you feel?”, “How is your pain?” and “Does this taste good?” Such a device may also be adapted to present interrogatory questions related to observational data, such as “What color is the sky?” or “Is it sunny outside?” The device can prompt the patient and record responsive data from the patient using visual and/or audible cues. For example, the patient can press coded response buttons or type an appropriate response on a keypad. Alternatively, responsive data may be collected by allowing the patient to speak into a microphone and using speech recognition software to process the response.

In some examples, the remote server system 108 comprises one or more computers, such as a database server 114, a messaging server 116, a file server 118, an application server 120 and a web server 122. The database server 114 is configured to provide database services to clients, which may be other servers in the remote server system 108. The messaging server 116 is configured to provide a communication platform for users of the remote server system 108. For example, the messaging server 116 may provide an email communication platform. Other types of messaging, such as short message service (SMS), instant messaging, or paging services. The file server 118 can be used to store documents, images, and other files for the web server 122 or as a general document repository. The application server 120 can provide one or more applications to the web server 122 or provide client-server applications to the client terminals 126. To enable some of these services provided by these servers 114, 116, 118, 120, and 112, the remote server system 108 can include an operations database 124. The operations database 124 can be used for various functions and may be composed of one or more logically or physically distinct databases. The operations database 124 can be used to store clinician data for individual patients, patient populations, patient trials, and the like. In addition, the operations database 124 can be used to store patient data for individual patients, patient populations, patient trials, and the like. For example, the operations database 124 may include a copy of, a portion of, a summary of, or other data from an electronic medical records system. In addition, the operations database 124 can store device information, such as device settings for a particular patient or a group of patients, preferred device settings for a particular clinician or a group of clinicians, device manufacturer information, and the like. In addition, the operations database 124 can be used to store raw, intermediate, or summary data of patient indications along with probabilistic outcomes (e.g., a patient population profile and a corresponding 1-year survival curve).

In an example, one or more client terminals 126 are locally or remotely connected to the remote server system 108 via network 110. The client terminals 112 are communicatively coupled to the remote server system 108 using a connection 128, which may be wired or wireless in various examples. Examples of client terminals 126 may include personal computers, dedicated terminal consoles, handheld devices (e.g., a personal digital assistant (PDA) or cellular telephone), or other specialized devices (e.g., a kiosk). In various examples, one or more users may use a client terminal 126 to access the remote server system 108. For example, a customer service professional may use a client terminal 126 to access records stored in the remote server system 108 to update patient records. As another example, a physician or clinician may use a client terminal 126 to receive or provide patient-related data, such as comments regarding a patient visit, physiological data from a test or collected by a sensor or monitor, therapy history (e.g., IMD shock or pacing therapy), or other physician observations.

In some examples, the IMD 102 is adapted to store patient data and to use the data to provide tailored therapy. For example, using historical physiological data, an IMD 102 may be able to discriminate between lethal and non-lethal heart rhythms and deliver an appropriate therapy. However, it is often desirable to establish a proper baseline of historical data by collecting a sufficient amount of data in the IMD 102. In some examples, a “learning period” of some time (e.g., thirty days) is used to establish the baseline for one or more physiological signals. An IMD 102 may, in an example, store a moving window of data of operation, such as a time period equal to the learning period, and may use the information as a baseline indication of the patient's biorhythms or biological events.

Once the baseline is established, then acute and long-term patient conditions may be determined probabilistically. The baseline may be established by using historical patient records or by comparing a patient to a population of patients. In an example, a diagnostic technique uses a patient-based baseline to detect a change in a patient's condition over time. Examples of a diagnostic technique that uses a patient-derived baseline are described in the next section.

In an example, patient diagnostics are automatically collected and stored by the implanted device 102. These values may be based on the patient's heart rate or physical activity over a time period (e.g., 24-hour period) and each diagnostic parameter is saved as a function of the time period. In one example, heart-rate based diagnostics utilize only normal intrinsic beats. For heart rate variability (HRV) patient diagnostics, the average heart rate can be found at each interval within the time period, for example, at each of the 288 five-minute intervals occurring during 24 hours. From these interval values, the minimum heart rate (MinHR), average heart rate (AvgHR), maximum heart rate (MaxHR) and standard deviation of average normal-to-normal (SDANN) values may be calculated and stored. In one example, the implanted device 102 computes a HRV Footprint® patient diagnostic that can include a 2-dimensional histogram that counts the number of daily heartbeats occurring at each combination of heart rate (interval between consecutive beats) and beat-to-beat variability (absolute difference between consecutive intervals). Each histogram bin contains the daily total for that combination. The percentage of histogram bins containing one or more counts can be saved each day as the footprint percent (Footprint %). The implanted device 102 can also provide an Activity Log® patient diagnostic (Activity %), which can include a general measure of patient activity and can be reported as the percentage of each time period during which the device-based accelerometer signal is above a threshold value.

### Example Operations

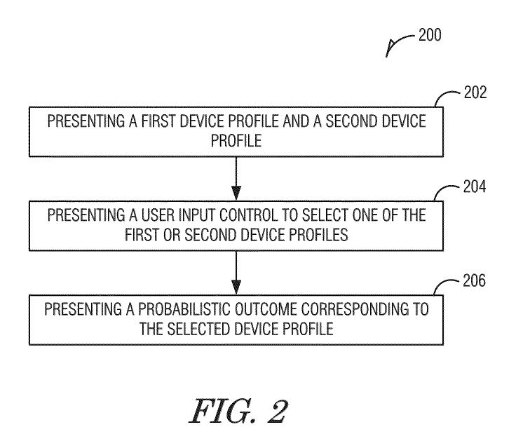

FIG. 2 is a flow chart illustrating a method 200 for presenting information to assist a clinician when programming a medical device. At 202, a first device profile and second device profile are presented in a graphical user interface (GUI), where the GUI is displayed in an electronic display of a computing device. In an example, the first and second profiles each include at least one device parameter used to configure a medical device of a subject. In an example, the first device profile is a current device profile of the medical device and the second device profile is a recommended device profile for the medical device. In an example, the recommended device profile is obtained from a database of device profiles. In an example, the computing device is a device programmer.

At 204, a user input control is presented in the GUI, where the user input control is to select one of the first or second device profiles to provide a selected device profile. The user input control can be one or more of varying types of conventional user input controls, such as, for example, a radio selection group, a check box selection group, a list, a slider bar, or a text input.

At 206, a probabilistic outcome of the subject corresponding to the selected device profile is presented in the GUI. The probabilistic outcome can be presented in various ways, such as, for example, a bar chart, a line graph, a text table, a pie graph, or a pictograph. Other types of presentations may be used, such as multimedia presentations (e.g., video or animated graphics) or audio presentations. In an example, the probabilistic outcome is displayed with the selected device profile in the GUI.

In an example, a user input control to configure the medical device using the selected device profile is presented in the GUI. In response to the user input control being activated, the medical device is configured with the selected device profile.

In an example, a user input control to adjust a parameter associated with the selected device profile is presented in the GUI. In response to the user input control being used, the probabilistic outcome is dynamically revised to provide a revised outcome. The revised outcome is then presented in the graphical user interface.

In an example, a user input control to select the probabilistic outcome from a plurality of available probabilistic outcomes is presented in the GUI. For example, available probabilistic outcome may include a number of shocks, right ventricle (RV) pacing percentage, atrial fibrillation (AF) burden, stroke event, etc. The plurality of available probabilistic outcomes are selected based on at least one of the first or second device profiles. In a further example, the medical device is identified to provide a medical device identification and the medical device identification is used to selectively present the plurality of available probabilistic outcomes. For example, some outcomes may not be relevant or available for some devices or device settings. In addition, in some cases, a probabilistic outcome is not as useful as another probabilistic outcome. To increase the visual efficacy of the presentation, fewer more relevant outcomes may be preferable to a greater number of less relevant outcomes. Other mechanisms may be used to pare down or selectively present a list of available outcomes. For example, outcomes may be filtered using one or more of the medical device in use, the programming profile, the patient's indications, or a clinician's preferences.

In an example, a user input control to select a second probabilistic outcome is presented. After a second probabilistic outcome is selected, the second probabilistic outcome of the subject corresponding to the selected device profile is presented. In a further example, the second probabilistic outcome is presented with the first probabilistic outcome.

In an example, after programming a device, the device and the patient with the device are monitored. Monitoring can be performed at the device level or at a system level. For device-level monitoring, the device itself can periodically check various physiological indications and cross-reference with device history to determine the efficacy of the adjusted programming settings. At a system level, the device may regularly or periodically report data to a system, and the system can then perform analysis on the patient history and the device history to determine whether the adjusted settings are providing an improved experience for the patient. Should a patient outcome deviate too far from a target outcome, such as by violating a threshold value or condition, an alert can be communicated. The alert may be communicated to an attending clinician, an electronic system (e.g., a data warehouse, an electronic medical records database, or other healthcare system), or to other parties, such as the patient or the patient's family. As such, by monitoring adjusted programming parameters and their effect on a patient or device, a clinician can be more confident when implementing system-provided recommended settings with the knowledge that when programming parameters are detected to be less effective than targeted, the system will alert the clinician and changes can be made proactively.

In an example, when a user (e.g., a physician, clinician, or other healthcare provider) selects an outcome to view, the selection is recorded. The history of previously-selected outcomes can then be used in various ways to assist the user during subsequent uses of the GUI. For example, using the history of previous selections, more-frequently used outcomes may be displayed by default. As another example, more-frequently used outcomes may be presented higher in an option list, such as in a dropdown list of available outcomes. While the history of selected outcomes is one way to capture a user's preference, another more direct manner includes setting one or more user preferences, where the preferences indicate the preferred outcomes. In this manner, for example, a set of one or more outcomes may be selected to be default outcomes, which are then presented in the GUI automatically. Similarly, a user may weight certain outcomes such that if outcomes with higher weights are available, then they are displayed preferably to those with lower weights. As users may have preferences for some outcomes over others to base their decisions on, historical usage or express user preferences may be used to facilitate quicker decisions and provide more confidence to the user.

### Example User Interfaces

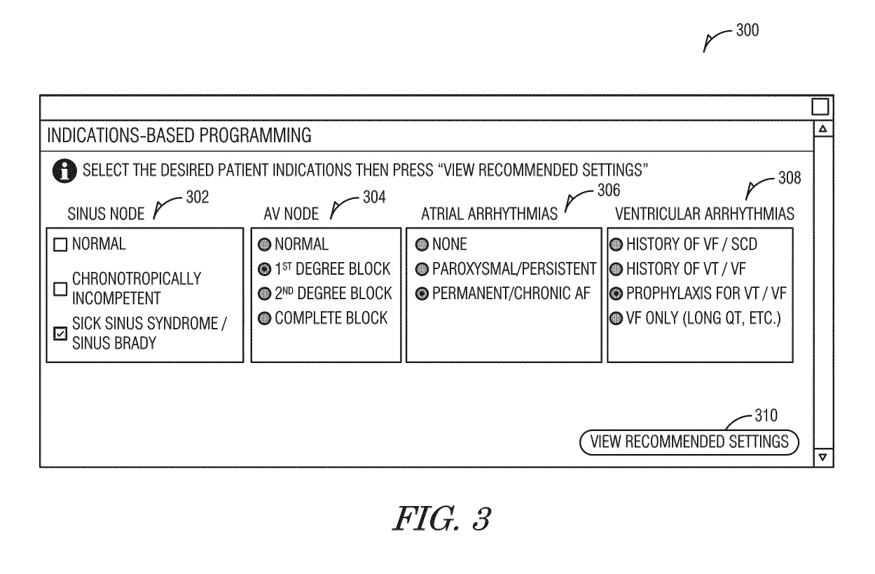

FIGS. 3-6 are diagrams illustrating example user interfaces. Although specific user interfaces are represented, it is understood that other user interfaces that provide the same or similar functionality are encompassed in the scope of this description.

FIG. 3 is a diagram illustrating an example user interface 300 that may be provided to user to collect patient indications. The user interface (UI) 300 includes one or more categories of indications. In the example shown, the UI 300 includes a “Sinus Node” category 302, an “AV Node” category 304, an “Atrial Arrhythmias” category 306, and a “Ventricular Arrhythmias” category 308. A user, such as a clinician or attending physician, can select one or more indications from one or more categories to provide information characterizing a patient's condition. This may be done once, such as during an initial implant or device activation phase, or more than once, such as during follow up visits or ongoing therapy. While some categories include indications that are mutually exclusive (e.g., the AV Node category 304 provides for four mutually exclusive options) other categories may include indications that can occur in combination or contemporaneously (e.g., the Sinus Node category 302 provides for various sinus conditions, some of which may be present in combination). In an example, indications in one or more categories are filtered or sorted. Filtering may be based on a particular patient's condition, a patient population, or a clinician's preference. Sorting may be based on relevance or likelihood of occurrence. For example, a particular patient may be compared to a patient population and a ranking of indications can be determined by analyzing the rate of occurrence in the population of persons similar to the patient. In addition, patient indications may be automatically obtained from a patient database, such as an electronic medical records database. In such an example, a user may not have to select the indications for a particular patient. Indications for a patient can be saved in a database and appear in the UI after identifying the patient.

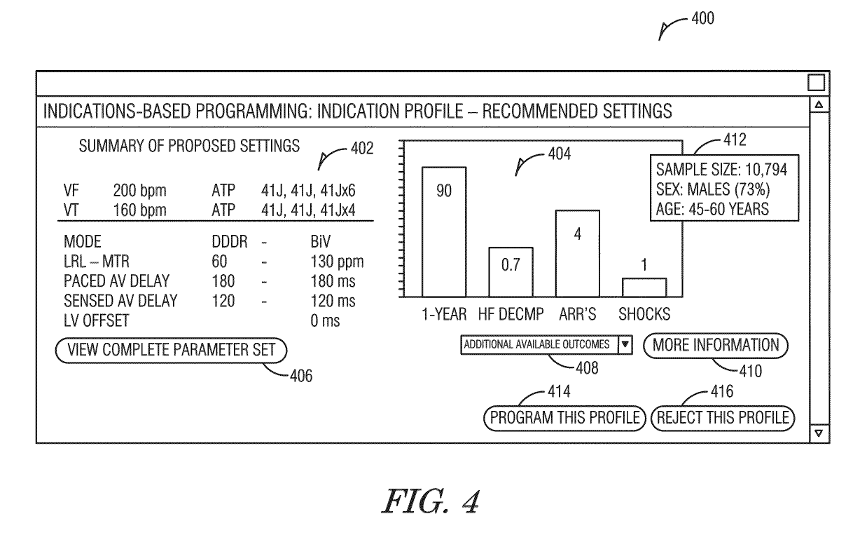

Once the user has entered indications, a View Recommended Settings control 310 may be activated. In an example, the View Recommended Settings control 310 triggers a navigation from the UI 300 to another UI, such as the one illustrated in FIG. 4 (discussed below). In an example, the View Recommended Settings control 310 operates to activate a pop-up window or other interface, within which summary information is displayed.

Although FIG. 3 illustrates some patient indications, it is understood that any patient indication can be included in the UI 300, including related data such as comorbidities.

FIG. 4 is a diagram illustrating an example user interface 400 to display recommended device settings. The UI 400 may be displayed after the View Recommended Settings control 310 (FIG. 3) is activated, although the UI 400 may be generated as a result of other user interface operations as well. The UI 400 includes a summary of proposed settings 402 and a presentation of one or more probabilistic outcomes 404.

The summary of proposed settings 402 may include a subset of parameters used to program a device. In this case, the UI 400 includes a View Complete Parameter Set control 406 to navigate to a presentation of the complete parameter set. Alternatively, the summary of proposed settings 402 may provide all of the parameters. For example, using a scrollable sub-window or a frame, or other formatting techniques, a lengthy list of parameters may be displayed in a compact form.

The presentation of probabilistic outcomes 404 can include one or more probabilistic outcomes based on the proposed settings. Examples of probabilistic outcomes include, but are not limited to, 1-year survival rate, heart failure (HF) decompensation events over time (e.g., per month or per year), number of sustained arrhythmia episodes over time (e.g., per year), number of shocks in a time period, and BiV pacing percentage. In addition, outcomes may be derived from or related to a patient's disease, device, or other patient condition. In the example shown, the presentation of probabilistic outcomes 404 includes a 1-year survival rate of 90%, a HF decompensation events per month of 0.7, a number of sustained arrhythmia episodes per month of 4, and a number of shocks per year of 1. Although four probabilistic outcomes are displayed in this example, more or less may be used. For example, a default set of outcomes may be used initially. After a time, a user may modify the displayed outcomes such that certain ones are always displayed or never displayed based on various preferences. In addition, outcomes may be displayed based on the context. A list of additional available outcomes control 408 may be used to select a particular outcome, then a More Information control 410 can be activated to display detailed information about the outcome, which may be displayed along with the corresponding device profile. In an example, the selected outcome from the list of additional available outcomes control 408 is displayed in the presentation of probabilistic outcomes 404. In another example, the outcome selected from the list of additional available outcomes control 408 is displayed in a separate interface, such as a pop-up window. An example of such an interface is illustrated in FIG. 5.

In addition, the UI 400 includes a legend 412. In this example, pertinent population data is presented to the user. Such information may be used to further validate the proposed settings and provide a level of confidence to the user that the settings are appropriate for the situation.

The UI 400 also includes a Program this Profile control 414 and a Reject this Profile control 416. Using the Program this Profile control 414 can program the device using the recommended settings. Should the user decide to reject the settings, activating the Reject this Profile control 416 can discard the current settings, which may also inhibit the settings from being presented again.

FIG. 5 is a diagram illustrating an example user interface 500 to display detailed information about an outcome. In the example shown, the survival curve outcome is presented. The UI 500 includes a graph over time of the selected outcome 502. In this case, the survival probability is calculated from of a subset of a population similar to the patient with the recommended programming settings, or with programming settings substantially similar to the recommended settings. The UI 500 also includes an alternative profile interface 504 to add, remove, or modify alternative programming options. In the example shown, two alternative programming profiles have been created: Alternative 1 and Alternative 2. Each alternative profile is represented on the graph 502, as depicted in the legend 506. The alternative profiles include modifications from the recommended profile, which acts as a baseline or a reference profile. The parameters that differ for each alternative profile are displayed in the alternative profile interface 504.

To modify an existing alternative profile, the user may activate the ‘+’ control 508. Doing so may navigate the user to a parameter selection screen where one or more parameters may be added, removed, or revised for the selected alternative profile.

To remove an alternative profile, the user may activate the ‘x’ control 510. Upon activating the ‘x’ control 510 for a particular alternative profile, the corresponding alternative profile is removed from the graph 502 and the alternative profile interface 504.

It is understood that the ‘+’ control 508 and the ‘x’ control 510 may be implemented using other types of user interface controls, such as a hyperlink, a script object, or an hypertext markup language (HTML) object.

To add a new alternative profile, the Add Alternative control 512 can be used. When creating an alternative profile, the user may be presented with a parameter selection screen similar to that used to add, remove, or revise a parameter in an existing alternative profile, except that the initial values for each parameter are based on the settings of the recommended profile.

By using the UI 500 as illustrated in FIG. 5, a user can quickly and easily compare the probabilistic outcome of a subject according to one or more device profiles.

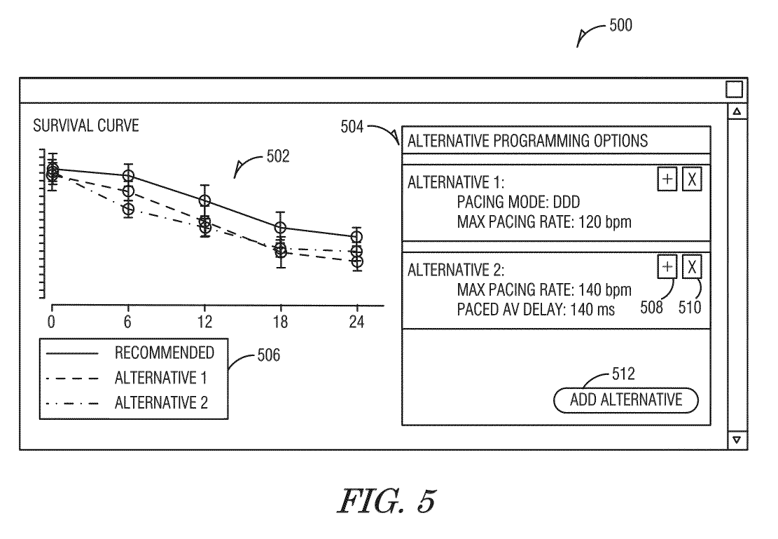

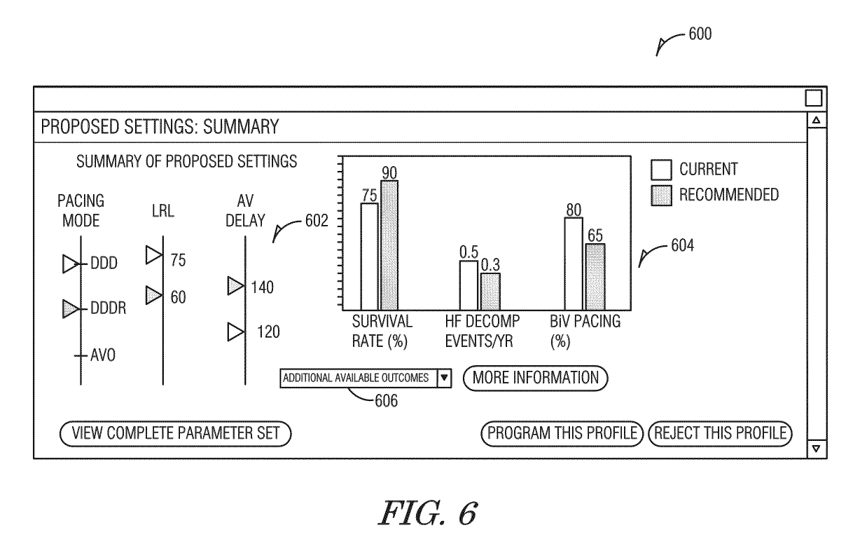

FIG. 6 is a diagram illustrating an example user interface 600 to display recommended device settings. Where the UI 400 to display recommended settings as illustrated in FIG. 4 may be used at an initial implant or other initial phase of a device installation or implementation, the UI 600 as illustrated in FIG. 6 can be used to compare an existing device profile with proposed or recommended device profile. The UI 600 includes input controls 602 to modify parameters and a presentation of outcomes 604 for comparing profiles. A user may interact with the various input controls 602, here illustrated as slider controls, to adjust individual parameters. As a user modifies one or more parameters, the displayed outcomes 604 can be dynamically updated to reflect updated probabilistic outcomes. As with FIG. 4, a list of additional available outcomes is provided in a control 606, which when used can add the additional selected outcome to the presentation of outcomes 604 and/or provide information about the additional selected outcome in a separate display (e.g., a pop-up window, a sub-frame, or the like).

Although FIG. 6 illustrates a comparison between a current device profile and a recommended device profile, it is understood that the comparison could be between any two device profiles, such as between a recommended profile and a user-generated profile, between two system-generated profiles (e.g., a first recommended profile and a second recommended profile), or between two user-generated profiles.

In UI 600, as with the UI 400 illustrated in FIG. 4, the user may use UI controls to view a complete parameter set, view specifics about a particular outcome, program a device with the recommended profile settings, or reject the profile settings.

While the probabilistic outcomes are useful when comparing device profiles, other mechanisms may be used to provide further utility. As an example, a situation may occur where one programming profile may provide for an increased benefit to one outcome at the expense of another outcome, while conversely another programming profile may provide for an inverse benefit or a different beneficial result. For instance, one programming profile may provide the best probability in reducing the number of shocks, but does not produce the best AF burden number. Another profile may produce a better AF burden number, but may not provide the best survivability. The large number of outcomes and their interrelationships makes it difficult to compare programming profiles.

In an example, a user may provide one or more weighted values or weights to each probabilistic outcome. Using the weights assigned to the outcomes, an outcome score can be calculated. The UI 600 can then display an outcome score based on the user's preferences, as represented by the weights. By comparing the outcome scores, which represent the combined weighted outcomes, a user can more easily compare the corresponding device programming profiles. The user can then revise either the programming parameters in a particular profile or the weights assigned to one or more outcomes to modify the outcome score for a particular programming profile.

In an example, default weights may be provided. Default weights may be system-generated or generated externally. For example, a system-generated default weight may be derived from a patient population analysis or a particular patient history. External sources of default weights may include medical boards, advisory boards, peer reviews, or the like. Although the use of weights is described, it is understood that other types of calculations, aggregations, or representations of outcomes can be used. For example, the ratio of different outcomes may be used to rank, sort, or aggregate outcomes. As another example, thresholds may be used to filter programming profiles by the probabilistic outcomes they have a chance of producing.

While FIGS. 4-6 illustrate providing outcome-based projections, another way to provide insight into suggested or recommended programming profiles is at the parameter level. For example, in FIG. 6, while a combination of parameters may affect a particular outcome, there is no way for a user to ascertain whether a particular parameter is within an acceptable range. Although the input controls 602 of FIG. 6 or the parameter selection screen described in the context of FIG. 5 may be adapted to constrain parameter values to known good or acceptable ranges (e.g., based on community standards, statistical analysis, or user preferences), there are other mechanisms to provide additional insight.

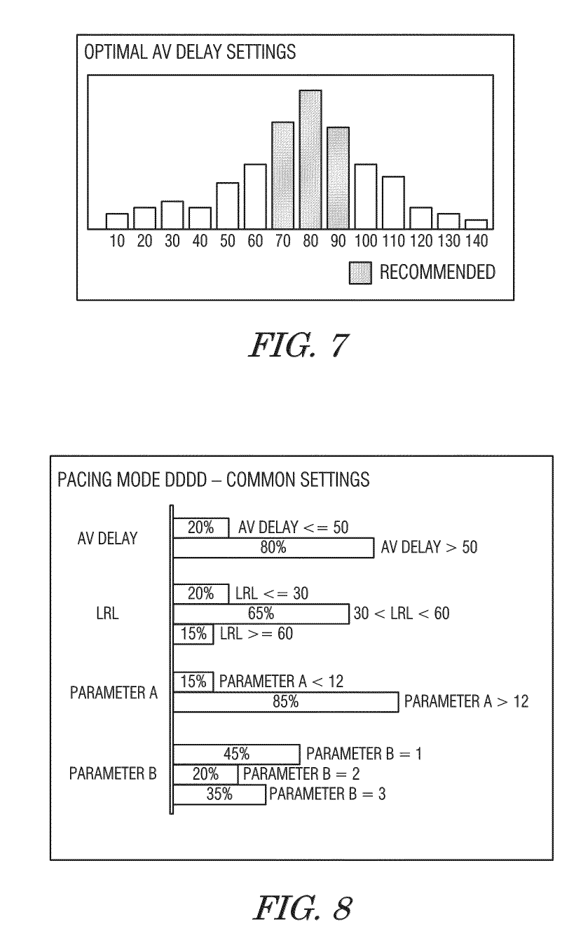

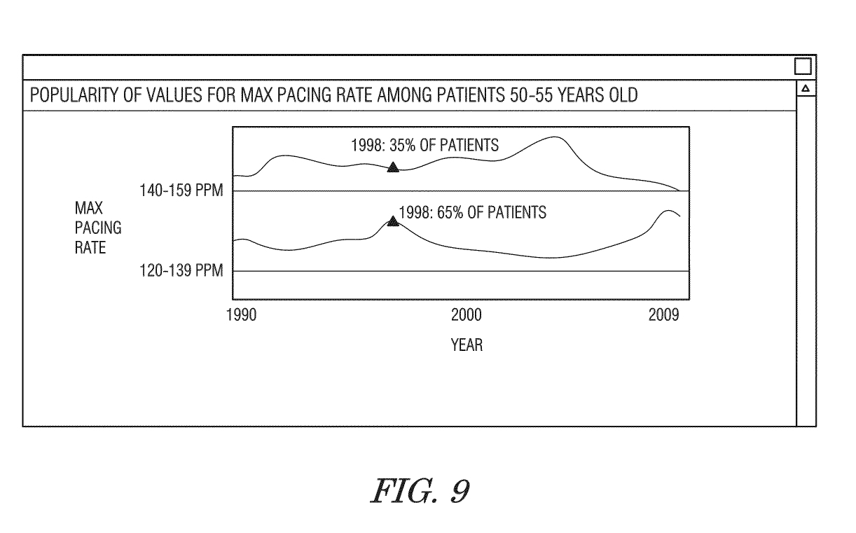

FIGS. 7 and 8 are diagrams illustrating population-based views of parameter usage. For example, in FIG. 7, the distribution of a particular parameter value may be presented along with an indication of where the recommended parameter value occurs in the distribution. Seeing this, the user is more assured that the recommended setting is accurate and within an acceptable range. Similarly, in FIG. 8, parameter values for a particular pacing mode are presented. The recommended values (not shown) may be placed in proximity to the bar charts of FIG. 8 to provide a reference for the user. FIG. 9 is a diagram illustrating a temporal view of parameter usage. The use of a particular parameter value over time may be useful, for example, to see how a medical community's importance of a particular parameter value has changed over time. It is understood that any of the view in FIGS. 7-9 may be implemented in the user interfaces 400, 500, or 600.

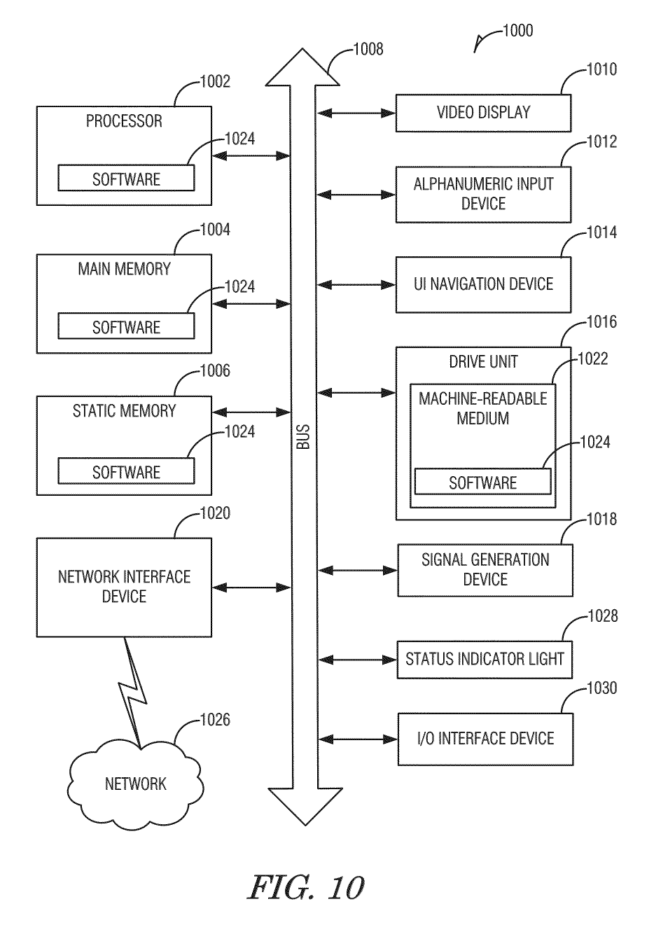

FIG. 10 is a block diagram of machine in the example form of a computer system 1000 within which a set of instructions, for causing the machine to perform any one or more of the methodologies discussed herein may be executed. In alternative examples, the machine operates as a standalone device or may be connected (e.g., networked) to other machines. In a networked deployment, the machine may operate in the capacity of a server or a client machine in server-client network environment, or as a peer machine in a peer-to-peer (or distributed) network environment. The machine may be a server computer, a client computer, a personal computer (PC), a tablet PC, a Personal Digital Assistant (PDA), a cellular telephone, a web appliance, a device programmer, a repeater, or any machine capable of executing a set of instructions (sequential or otherwise) that specify actions to be taken by that machine. Further, while only a single machine is illustrated, the term “machine” shall also be taken to include any collection of machines that individually or jointly execute a set (or multiple sets) of instructions to perform any one or more of the methodologies discussed herein.

The example computer system 1000 includes a processor 1002 (e.g., a central processing unit (CPU) a graphics processing unit (GPU) or both), a main memory 1004 and a static memory 1006, which communicate with each other via a bus 1008. The computer system 1000 may further include a video display 1010 (e.g., a liquid crystal display (LCD) or a cathode ray tube (CRT)). The computer system 1000 also includes an alphanumeric input device 1012 (e.g., a keyboard), a user interface navigation device 1014 (e.g., a mouse), a disk drive unit 1016, a signal generation device 1018 (e.g., a speaker) and a network interface device 1020.

The disk drive unit 1016 includes a machine-readable medium 1022 on which is stored one or more sets of instructions (e.g., software 1024) embodying any one or more of the methodologies or functions described herein. The software 1024 may also reside, completely or at least partially, within the main memory 1004 and/or within the processor 1002 during execution thereof by the computer system 1000, the main memory 1004 and the processor 1002 also constituting machine-readable media. The software 1024 may further be transmitted or received over a network 1026 via the network interface device 1020.

While the machine-readable medium 1022 is shown in an example embodiment to be a single medium, the term “machine-readable medium” should be taken to include a single medium or multiple media (e.g., a centralized or distributed database, and/or associated caches and servers) that store the one or more sets of instructions. The term “machine-readable medium” shall also be taken to include any medium that is capable of storing, encoding or carrying a set of instructions for execution by the machine and that cause the machine to perform any one or more of the methodologies of the present invention. The term “machine-readable medium” shall accordingly be taken to include, but not be limited to, tangible media, such as solid-state memories, optical, and magnetic media.

### Additional Notes

The above detailed description includes references to the accompanying drawings, which form a part of the detailed description. The drawings show, by way of illustration, specific embodiments in which the invention can be practiced. These embodiments are also referred to herein as “examples.” Such examples can include elements in addition to those shown and described. The above description is intended to be illustrative, and not restrictive. For example, the above-described examples (or one or more aspects thereof) may be used in combination with each other. Other embodiments can be used, such as those identified by one of ordinary skill in the art upon review of the above description.

All publications, patents, and patent documents referred to in this document are incorporated by reference herein in their entirety, as though individually incorporated by reference. In the event of inconsistent usages between this document and those documents so incorporated by reference, the usage in the incorporated reference(s) should be considered supplementary to that of this document; for irreconcilable inconsistencies, the usage in this document controls.

In this document, the terms “a” or “an” are used, as is common in patent documents, to include one or more than one, independent of any other instances or usages of “at least one” or “one or more.” In this document, the term “or” is used to refer to a nonexclusive or, such that “A or B” includes “A but not B,” “B but not A,” and “A and B,” unless otherwise indicated. In the appended claims, the terms “including” and “in which” are used as the plain-English equivalents of the respective terms “comprising” and “wherein.” Also, in the following claims, the terms “including” and “comprising” are open-ended, that is, a system, device, article, or process that includes elements in addition to those listed after such a term in a claim are still deemed to fall within the scope of that claim. Moreover, in the following claims, the terms “first,” “second,” and “third,” etc. are used merely as labels, and are not intended to impose numerical requirements on their objects.

The Abstract is provided to comply with 37 C.F.R. §1.72(b), to allow the reader to quickly ascertain the nature of the technical disclosure. It is submitted with the understanding that it will not be used to interpret or limit the scope or meaning of the claims. Also, in the above Detailed Description, various features may be grouped together to streamline the disclosure. This should not be interpreted as intending that an unclaimed disclosed feature is essential to any claim. Rather, inventive subject matter may lie in less than all features of a particular disclosed embodiment. Thus, the following claims are hereby incorporated into the Detailed Description, with each claim standing on its own as a separate embodiment. The scope of the invention should be determined with reference to the appended claims, along with the full scope of equivalents to which such claims are entitled.

What is claimed is:

1. A system comprising:
an electronic display;
a memory to store a first and second device profile, each profile comprising at least one device parameter used to configure a medical device of a subject; and
a processor, coupled to the memory and the electronic display, the processor configured to:
present, in a graphical user interface displayed in the electronic display, the first device profile and second device profile;
present, in the graphical user interface, a user input control to select one of the first or second device profiles to provide a selected device profile; and
present, in the graphical user interface, a probabilistic outcome of the subject corresponding to the selected device profile.

2. The system of claim 1, wherein the processor is configured to:
present, in the graphical user interface, a user input control to configure the medical device using the selected device profile; and
in response to the user input control being activated, configure the medical device with the selected device profile.

3. The system of claim 2, further comprising:
a patient monitor operatively coupled to the processor and configured to:
determine an efficacy of the medical device when configured with the selected device profile;
compare the efficacy of the medical device to a target outcome; and
when the efficacy of the medical device deviates more than a threshold value from the target outcome, communicating an alert.

4. The system of claim 1, wherein the first device profile is a current device profile of the medical device and the second device profile is a recommended device profile for the medical device.

5. The system of claim 4, wherein the processor is configured to obtain the recommended device profile from a database of device profiles.

6. The system of claim 1, wherein the processor is configured to:
present a user input control to adjust a parameter associated with the selected device profile; and
in response to the user input control being used, dynamically revise the probabilistic outcome to provide a revised outcome; and
present the revised outcome in the graphical user interface.

7. The system of claim 1, wherein the processor is configured to:
present a user input control to select the probabilistic outcome from a plurality of available probabilistic outcomes, wherein the plurality of available probabilistic outcomes are selected based on at least one of the first or second device profiles.

8. The system of claim 7, wherein the processor is configured to:
identify the medical device to provide a medical device identification; and
use the medical device identification to selectively present the plurality of available probabilistic outcomes.

9. The system of claim 1, wherein the processor is configured to:
present a user input control to select a second probabilistic outcome; and
present, in the graphical user interface, the second probabilistic outcome of the subject corresponding to the selected device profile.

10. The system of claim 9, wherein the second probabilistic outcome is presented with the first probabilistic outcome.

11. A method comprising:
presenting, in a graphical user interface displayed in an electronic display of a computing device, a first device profile and second device profile, the first and second device profiles each comprising at least one device parameter used to configure a medical device of a subject;
presenting, in the graphical user interface, a user input control to select one of the first or second device profiles to provide a selected device profile; and
presenting, in the graphical user interface, a probabilistic outcome of the subject corresponding to the selected device profile.

12. The method of claim 11, comprising:
presenting, in the graphical user interface, a user input control to configure the medical device using the selected device profile; and
in response to the user input control being activated, configuring the medical device with the selected device profile.

13. The method of claim 11, wherein the first device profile is a current device profile of the medical device and the second device profile is a recommended device profile for the medical device.

14. The method of claim 13, comprising obtaining the recommended device profile from a database of device profiles.

15. The method of claim 11, comprising:
presenting a user input control to adjust a parameter associated with the selected device profile; and
in response to the user input control being used, dynamically revising the probabilistic outcome to provide a revised outcome; and
presenting the revised outcome in the graphical user interface.

16. The method of claim 11, comprising:
presenting a user input control to select the probabilistic outcome from a plurality of available probabilistic outcomes, wherein the plurality of available probabilistic outcomes are selected based on at least one of the first or second device profiles.

17. The method of claim 16, comprising:
identifying the medical device to provide a medical device identification; and
using the medical device identification to selectively present the plurality of available probabilistic outcomes.

18. The method of claim 11, comprising:
presenting a user input control to select a second probabilistic outcome; and
presenting, in the graphical user interface, the second probabilistic outcome of the subject corresponding to the selected device profile.

19. The method of claim 18, wherein the second probabilistic outcome is presented with the first probabilistic outcome.

20. A machine-readable medium including instructions, which when executed on a machine, cause the machine to:
present, in a graphical user interface displayed in an electronic display of the machine, a first device profile and second device profile, the first and second device profiles each comprising at least one device parameter used to configure a medical device of a subject;
present, in the graphical user interface, a user input control to select one of the first or second device profiles to provide a selected device profile; and
present, in the graphical user interface, a probabilistic outcome of the subject corresponding to the selected device profile.
